(19) United States
(12) Patent Application Publication
(10) Pub. No.: US 2011/0124745 A1
(43) Pub. Date: May 26, 2011

(54) Switchable Anionic Surfactants and Methods of Making and Using Same

(76) Inventors: Philip G. Jessop, Kingston (CA); Liang Chen Guang, Gatineau (CA); Jitendra R. Harjani, Ottawa (CA)

(21) Appl. No.: 12/901,189

(22) Filed: Oct. 8, 2010

Related U.S. Application Data
(60) Provisional application No. 61/272,598, filed on Oct. 9, 2009.

(51) Int. Cl. B01D 17/04; C07C 69/76; B01D 17/00; B09C 1/02; B08B 3/14

(52) U.S. Cl. 516/67; 560/64; 516/135; 516/197; 405/128.7; 134/109

(57) ABSTRACT

Reversibly switchable negatively charged surfactants are provided. The anionic surfactant is formed in aqueous solution by providing an absence of CO₂ and is converted to a non-surfactant neutral state through exposure to CO₂. The anionic switchable surfactants are useful to stabilize emulsions. In the neutral state they are useful to separate immiscible liquids. The surfactants find uses in polymerization, the oil industry, remediation of oil-contaminated soil and recapture of oil contaminant, and formation of solid particles such as plastics and nanoparticles.

### RELATED APPLICATIONS

This application claims the benefit of the filing date of U.S. Provisional Patent Application No. 61/272,598, filed on Oct. 9, 2009, the contents of which are incorporated herein by reference in their entirety.

### FIELD OF THE INVENTION

The field of the invention is surfactants. More specifically, the field of the invention is anionic surfactants that can be reversibly converted between surfactant and non-surfactant forms.

### BACKGROUND OF THE INVENTION

Surfactants are widely used, for example, to create emulsions in otherwise immiscible liquids (e.g., oil and water), to stabilize foams of gases in liquids, and to stabilize suspensions of solids in liquids. However, removal of a traditional non-switchable surfactant after use is often difficult and can create enormous amounts of waste. For example, nanoparticles can be formed by reduction of a metal compound to elemental metal in the presence of reductant and a surfactant. The surfactant coats the surface with charged layers to inhibit nanoparticles from combining with each other and forming larger particles. After the synthesis, removal of the surfactant from the surface is difficult because of strong interactions between the solid surface and the surfactant. In the area of cleaning and biphasic reactions, breaking the emulsion is often difficult but if achieved would allow better separations and cleaner disposal or recycling of the liquids. In the area of suspension of solids such as latexes and mineral flotation, isolation of solids from an aqueous suspension is hindered by the presence of a surfactant.

Other researchers have explored the area of switchable surfactants by turning off the surfactant, when it is no longer needed, to enable easier removal of the surfactant. Current techniques include: photoelectron-switchable surfactants, thermal switching surfactants, photochemical switching surfactant, pH switchable surfactants and redox switchable surfactants. The primary limitations of the above literature methods are the amount of energy consumed and the amount of chemical waste generated. For example, given the opaque or light-scattering nature of many emulsions, a very strong light source is required to permeate an emulsion in photochemical or photoelectric switching. In photoelectric switching, the emulsion may also be a poor conductor thus requiring large potentials. In pH switchable systems, the pH changes are achieved by the addition of acids and bases, creating undesirable salt waste each time. Redox reactions also generate waste after each cycle, but in such cases the waste may include heavy metal waste due to the use of metals as redox-switchable portions of surfactant molecules or due to the use of metal-containing or metal-promoted oxidants or reductants.

A series of switchable surfactants activated by CO₂ and deactivated by a substantial absence of CO₂ is described in International Patent Application No. PCT/CA2006/001877 by Jessop (published as International Patent Application Publication No. WO 2007/056859). Those surfactants, when activated (or “switched an”), are cationic surfactants since they bear a positive charge. In many applications there remains a need for an anionic surfactant, especially involving the preparation of solid particles (plastics, nanoparticles, colloids, paint latexes). There is a need for anionic surfactants that are able to switch by application of a trigger from one form with a first set of physical properties to another form with a second and different set of physical properties. In particular, anionic surfactants that can be switched between one form that has good surface activity and another form that has substantially no surface activity are highly sought after.

### SUMMARY OF THE INVENTION

A first aspect of the invention provides a compound comprising a hydrophobic moiety and a heteroatom; wherein when an appropriate trigger is applied, the compound in aqueous solution reversibly switches between two states, a neutral state and an anionic state, that are distinguishable from one another by their surface activities; where the heteroatom is protonated in the neutral state, and the heteroatom is deprotonated and negatively charged in the anionic state; and wherein a first said trigger, for converting the neutral state to the anionic state in an aqueous solution that has little or no dissolved CO₂, is addition of a base to the aqueous solution; a second said trigger, for converting the neutral state to the anionic state in an aqueous solution that comprises dissolved CO₂, is depletion of CO₂ from the aqueous solution; and a third said trigger, for converting the anionic state to the neutral state, is the addition of CO₂ to the aqueous solution.

In an embodiment of this aspect depletion of CO₂ from the aqueous solution is obtained by: heating the aqueous solution; exposing the aqueous solution to air; exposing the aqueous solution to a gas or gases that has insufficient CO₂ content to convert the anionic state to the neutral state; flushing the aqueous solution with a gas or gases that has insufficient CO₂ content to convert the anionic state to the neutral state; or a combination thereof.

In an embodiment of this aspect, the anionic state further comprises a cationic counterion, which is Na⁺, K⁺, Rb⁺, Cs⁺, Li⁺, a transition metal ion, Hg⁺, U⁺, Cr⁺, Pb⁺, Pu⁺, NH₄⁺, NR₄⁺, NRH₃⁺, NR₂H₂⁺, or NR₃H⁺, where R is a lower alkyl group.

In an embodiment of this aspect, the hydrophobic moiety comprises a C₄-C₁₀₀ hydrocarbon chain. In certain embodiments, the hydrocarbon chain is substituted. In certain embodiments, the heteroatom is O, S, or Se. In embodiments of the invention, the compound comprises a hydrophobic moiety that is a hydrocarbon chain, a headgroup attached at one end of the hydrocarbon chain, and a heteroatom that is proximal to the headgroup. In certain embodiments of the invention, the headgroup is an aryl moiety. In some embodiments, the aryl moiety is a heteroaryl moiety comprising the heteroatom. In some embodiments, the headgroup is an aryl moiety that is substituted by the heteroatom or by a moiety including the heteroatom. In some embodiments, the aryl group is substituted by a —((C₁-C₄)-heteroatom) or a -(heteroatom-(C₁-C₄)) moiety. In some embodiments, the neutral state comprises a phenol and the anionic state comprises a phenolate. In certain embodiments, the compound in its neutral state is octyl 4-hydroxy-3-nitrobenzoate and in its anionic state is 2-nitro-4-(octyloxycarbonyl)phenolate.

In a second aspect, the invention provides an anionic surfactant comprising: a hydrophobic moiety and a heteroatom; wherein when an appropriate trigger is applied, the anionic surfactant in an aqueous solution reversibly switches between two states, an anionic surfactant state and a neutral state, that are distinguishable from one another by their surface activities; where the heteroatom is protonated in the neutral state, and the heteroatom is deprotonated and negatively charged in the anionic state; and wherein a first said trigger, for converting the anionic state to the neutral state, is addition of CO₂ to the aqueous solution; a second said trigger, for converting the neutral state to the anionic state in an aqueous solution that has little or no dissolved CO₂, is addition of a base to the aqueous solution; a third said trigger, for converting the neutral state to the anionic state in an aqueous solution that comprises dissolved CO₂, is depletion of CO₂ from the aqueous solution.

In an embodiment of the above aspect, the protonated heteroatom has a pKa that is within about 2.5 pH units of the pKa of H₂CO₃, which is 6.35. In certain embodiments of this aspect, the neutral state is a demulsifier.

In a third aspect, the invention provides a method for stabilizing an emulsion of an aqueous liquid and a water-immiscible liquid comprising: combining an aqueous liquid and a water-immiscible liquid to form a mixture; adding to the mixture a compound of claim 1 which is either (i) in its anionic state; or (ii) in its neutral state and is converted to its anionic state in situ; and agitating the mixture to form a stable emulsion.

In embodiments of this aspect, the compound in its anionic state is 2-nitro-4-(octyloxycarbonyl)phenolate (1A) and in its neutral state is octyl 4-hydroxy-3-nitrobenzoate (1N).

In a fourth aspect, the invention provides a method for breaking an emulsion in a mixture, comprising providing an emulsion of an aqueous liquid and a water-immiscible liquid, the emulsion including a compound as described in the first aspect of the invention in its anionic state; and exposing the emulsion to CO₂ to convert the compound from its anionic state to its neutral state.

In certain embodiments, the fourth aspect further provides separating the aqueous liquid from the water-immiscible liquid. Some embodiments of the fourth aspect further comprise mixing the compound in its neutral state with an aqueous liquid that comprises dissolved CO₂; eliminating substantially all CO₂ from the mixture; and reforming the anionic state of the compound. In some embodiments the aqueous liquid further comprises a base. In certain embodiments, the base is NaOH, NaHCO₃, or Na₂CO₃. In some embodiments of the fourth aspect, the compound in its anionic state is 2-nitro-4-(octyloxycarbonyl)phenolate (1A). In some embodiments of this aspect, the heteroatom is attached directly to the aryl moiety.

A fifth aspect of the invention provides a method of remediating soil that is contaminated with one or more hydrophobic chemicals, comprising contacting the contaminated soil with a liquid that comprises water and an anionic surfactant of the first aspect so that at least a portion of the hydrophobic chemical becomes associated with the liquid to form contaminated liquid; optionally separating the contaminated liquid from residual solid soil; contacting the contaminated liquid with CO₂, COS, or CS₂ to convert a substantial amount of the surfactant from its anionic form to its non-ionic form, resulting in a two-phase liquid mixture having a hydrophobic liquid phase comprising the one or more hydrophobic chemicals, and an aqueous liquid phase; and separating the hydrophobic layer from the aqueous layer. In some embodiments of the fifth aspect the compound in its anionic state is 2-nitro-4-(octyloxycarbonyl)phenolate (1A).

A sixth aspect of the invention provides a system for cleaning a hydrophobic contaminant from a solid material, comprising means for contacting a mixture of solid material and hydrophobic contaminant with a liquid that comprises water and an anionic surfactant of any embodiment of the first aspect so that at least a portion of the hydrophobic contaminant becomes associated with the liquid to form contaminated liquid; optionally, means for separating the contaminated liquid from residual solid material; means for contacting the contaminated liquid with CO₂ to convert a substantial amount of the surfactant from its anionic form to its non-ionic form, resulting in a two-phase liquid mixture having a hydrophobic liquid phase comprising the hydrophobic contaminant, and an aqueous liquid phase; and means for separating the hydrophobic liquid phase from the aqueous liquid phase. In some embodiments of the sixth aspect, the compound in its anionic state is 2-nitro-4-(octyloxycarbonyl)phenolate (1A).

### BRIEF DESCRIPTION OF THE DRAWINGS

Embodiments of the invention will now be described, by way of example, with reference to the accompanying drawings.

FIG. 1 shows a NMR spectrum for the product of the reaction scheme shown, namely compound (1N), octyl 4-hydroxy-3-nitrobenzoate.

FIG. 2 shows a photo of vials 1 to 11 where the treatment of the vial contents is summarized in Table 1. Note that the odd numbered vials are ON, and appear as clear yellow solutions, while the even numbered vials are OFF, and appear as suspensions of white solid precipitate in pale yellow liquid. Vials are capped and the cap and vial are wrapped in PARAFILM™. White cylinder-shaped magnetic stir bars are in each vial and are visible in some vials.

FIGS. 3A, 3B, and 3C show photos of three vials, which are fully described in Example 6D. Briefly, Vial A held a solution of compound (1A) in FIGS. 3A, 3B and 3C; vial C held a solution of (1N) in FIGS. 3A, 3B and 3C; and vial B held a solution that was converted between (1N) in FIG. 3A, to a mixture of (1N) and (1A) in FIG. 3B, and finally to a solution of (1A) in FIG. 3C.

### DETAILED DESCRIPTION OF THE INVENTION

Surfactants have many uses, including: (i) cleaning of materials; (ii) facilitating biphasic reactions by creating an emulsion between two liquids (the surface area of the interface between the liquids being increased causing the extent of reaction between reagents in the two liquids to be increased); (iii) protecting nanoparticles, nanomaterials, and other small particles from agglomeration or other undesirable reactions during their syntheses, (iv) facilitating flotation of certain minerals in water during the separation of some minerals from other minerals, (v) stabilizing emulsions of monomers; (vi) stabilizing suspensions of solid polymer particles during and after emulsion polymerizations, mini-emulsion polymerizations, micro-emulsion polymerizations and suspension polymerizations, (vii) stabilizing emulsions of oils during transportation by pipeline, (viii) stabilizing paints and coatings during preparation, storage, shipping and use, and (ix) stabilizing foams or suspensions. The following terms will be used herein:

### Definitions

As used herein, “air that has had its carbon dioxide component substantially removed” means that the air has insufficient carbon dioxide content to interfere with the removal of carbon dioxide from the solution. For some applications, untreated air may be successfully employed, i.e., air in which the carbon dioxide content is unaltered; this would provide a cost saving.

As used herein, “aliphatic” refers to hydrocarbon moieties that are linear, branched or cyclic, may be alkyl, alkenyl or alkynyl, and may be substituted or unsubstituted. “Aryl” means a moiety including a substituted or unsubstituted aromatic ring, including heteroaryl moieties and moieties with more than one conjugated aromatic ring; optionally it may also include one or more non-aromatic ring. Examples of aryl moieties include, phenyl, biphenyl, naphthyl, anthryl, phenanthryl, pyrenyl, pyridyl, bipyridyl, xylyl, indolyl, thienyl, and quinolinyl.

As used herein “unsubstituted” refers to any open valence of an atom being occupied by hydrogen. Also, if an occupant of an open valence position on an atom is not specified then it is hydrogen.

“Substituted” means having one or more substituent moieties whose presence does not interfere with the desired reaction. Examples of substituents include alkyl, alkenyl, alkynyl, halide, aryl, aryl-halide, heteroaryl, cyclyl (non-aromatic ring), Si(alkyl)₃, Si(alkoxy)₃, halo, alkoxyl, amino, amide, hydroxyl, thioether, alkylcarbonyl, alkylcarbonyloxy, arylcarbonyloxy, alkoxycarbonyloxy, a ryloxycarbonyloxy, carbonate, alkoxycarbonyl, aminocarbonyl, alkylthiocarbonyl, phosphate, phosphate ester, phosphonato, phosphinato, cyano, acylamino, imino, sulfhydryl, alkylthio, arylthio, thiocarboxylate, dithiocarboxylate, sulfate, sulfato, sulfamoyl, sulfonamide, nitro, nitrite, azido, heterocyclyl, ether, ester, silicon-containing moieties, thioester, or a combination thereof. Preferable substituents are alkyl, aryl, heteroaryl, ether, and combinations thereof.

As used herein, “heteroatom” refers to non-hydrogen and non-carbon atoms, such as, for example, O, S, and Se. Preferred heteroatoms that are useful according to the invention can be protonated.

For the purposes of this description, a compound that switches between two states is a convenient description of a pair of almost identical compounds differing only in that one compound is protonated at a particular site and the other compound is deprotonated at that site.

As used herein, “pKa” is equal to −log Kₐ, where Kₐ is an acid dissociation constant. An acid dissociation constant is a quantitative measure of the strength of an acid in solution and it is the equilibrium constant for dissociation in the context of acid-base reactions.

“Alcohol” means a molecule of the formula ROH, where R is alkyl, alkenyl, alkynyl, aryl, silyl, or siloxyl, and may be linear, branched, cyclic, and may be substituted or unsubstituted. Substituents are as defined above and include moieties that do not interfere with the desired reaction. Alcohols for use in the invention may optionally be chiral.

“Switchable” means able to be converted from a first state with a first set of physical properties to a second state with a second set of physical properties, wherein one or more of said properties is different between the first and second sets. A “trigger” is a change of reaction conditions (e.g., introduction or removal of a gas, change in temperature) that causes a change in the physical property or properties.

As used herein, the term “reversible” means that the reaction can proceed in either direction (backward or forward) depending on the reaction conditions.

As used herein, “short chain aliphatic” or “lower aliphatic” refers to C₁ to C₄ aliphatic.

As used herein, “long chain aliphatic” or “higher aliphatic” refers to C₅ to C₂₅ aliphatic.

As used herein, “ionic” means containing or involving or occurring in the form of positively or negatively charged ions, i.e., charged moieties. “Zwitterionic” means having at least two oppositely charged groups present at different locations within the same molecule.

As used herein, “NMR” means Nuclear Magnetic Resonance.

As used herein, “wet diethyl ether” means diethyl ether that has been exposed to the atmosphere such that water from surrounding air has entered the diethyl ether.

As used herein, “immiscible” means unable to merge into a single phase. Thus two liquids are described herein as “immiscible” if they form two phases when combined in a proportion. This is not meant to imply that combinations of the two liquids will be two-phase mixtures in all proportions or under all conditions.

As used herein, “contaminant” refers to a compound or mixture of compounds whose removal is desirable and is not meant to imply that the compound or mixture has no value. For example, the contaminant may be oil which is removed and recaptured from tar sands, and the oil has economic value.

As used herein, the term “compound (1N)” (where N represents “neutral”) is octyl 4-hydroxy-3-nitrobenzoate, whose structural formula is shown below:

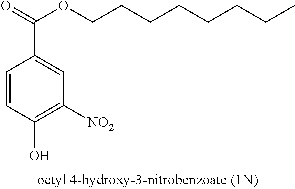

In aqueous solutions of sufficiently basic pH levels, (1N) becomes deprotonated and forms “compound (1A)” (where A represents “anionic”). Compound (1A) is the anion 2-nitro-4-(octyloxycarbonyl)phenolate, whose structural formula is shown below:

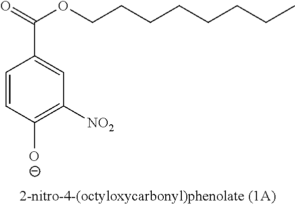

In solution, compound (1A) would have a cationic counterion, which is not H⁺, associated with it, as discussed further below.

As used herein, “ON” and “OFF” are terms used to indicate which of two forms has greater surface activity (indicated by “ON”) or less surface activity (indicated by “OFF”). This is not meant to imply that the “OFF” form has no surface activity. The “OFF” form may be surface inactive, it may be a demulsifier, or it may be a surfactant that is merely different in properties from the form that is called “ON”.

As used herein the term “emulsion” means a fine dispersion of minute droplets of one liquid in another liquid in which it is immiscible. As used herein, the term “suspension” means a mixture of two substances, one of which is finely divided and dispersed in the other.

As used herein, the term “hydrophobic” means having little affinity for water.

Surfactants are important additives in mixtures of liquids of differing hydrophobicities, e.g., mixtures of oil and water. When such liquids are mixed with a surfactant present, a stable emulsion forms. As used herein, a “stable emulsion” means an emulsion that lasts either indefinitely or at least as long as required for a particular application or use. Surfactants act at the interface between such liquids and keep the emulsion from separating into distinct layers. Stabilizing the emulsion is a positive trait of the surfactant when the emulsion is desired. However, once the emulsion is no longer desired, the presence of a traditional non-switchable surfactant can be problematic. (For example, the emulsion has been transported, perhaps by pipeline, and it is now desirable to separate its component.) Since traditional surfactants cannot be turned off, separating the emulsion into its component liquids can be time consuming, energy intensive and costly.

Surfactants are important additives in suspensions of solids in liquids, e.g., solid polymer particles in water. With a surfactant present, a stable suspension is possible when fine particles are formed in water or are mixed into water. Such stability is possible since surfactants act at the interface between such fine particles and the liquid, thereby preventing or slowing the processes by which solid particles tend to separate from liquids (i.e., flocculating, settling, coagulating, clumping, floating upwards, or otherwise gathering). Stabilization of suspensions is a positive trait of the surfactant when the suspension is desired; however, once the suspension is no longer desired the presence of a traditional non-switchable surfactant can be problematic. An example of a time when it is desirable to have a surfactant present is when a latex suspension has been formed by emulsion polymerization. The stable emulsion that is made possible by the presence of a surfactant allows the polymerization to take place, forming the latex suspension. The suspension itself is then kept stable by the surfactant, allowing the suspension to be shipped and stored. However, when it becomes desirable to separate its components, separating the solid particles from the liquid can be time consuming, energy intensive and costly because traditional surfactants cannot be turned off.

For clarity herein, the neutral form is denoted as OFF, meaning it has significantly less or no surface activity. Similarly, the anionic form is denoted as ON because it has greater relative surface activity. ON and OFF are terms that refer to the surfactant-properties of the state of the compound.

In contrast to mixtures stabilized by traditional non-switchable surfactants, a mixture stabilized by an anionic surfactant of the invention advantageously can be readily separated into its components after application of a trigger. Once triggered, the switchable anionic surfactant is switched to its OFF state. By switching the surface activity from ON to OFF, the surface activity is lessened. The emulsion or suspension, which is now no longer stabilized by a competent surfactant, separates readily. Such switchable anionic surfactants may be used in many applications such as, for example, emulsion polymerization, latex formulation, cleaning products, chemical manufacturing, oil sands separations and/or separation of tailings ponds, mining, resolution and separation of chemical compounds, polymer separation, polymer compositions, cosmetics, paints and coatings, chemical recycling, water treatment, dyes and pigments, chemical signaling, environmental remediation, targeted delivery of chemical agents, targeted delivery of biological agents, controlled release of chemical agents, controlled release of biological agents, self-assembly of nano-scale materials, and ore flotation.

In most of the discussion herein, the term “CO₂” will be employed, though that gas may in some circumstances optionally be replaced by or mixed with carbon disulfide (CS₂) or carbonyl sulfide (COS). Carbon disulfide is not preferred because of its flammability, its toxicity, and its negative impact on the environment. Carbonyl sulfide is not preferred because of its flammability, its negative impact on human health (irritant, damage to nervous system), and its negative impact on the environment. Nevertheless, CS₂ and COS are expected to be capable of triggering the same change in the switchable anionic surfactant as can carbon dioxide.

A trigger to switch anionic surfactants according to the invention from one state to another is the presence or substantial absence of CO₂. Carbon dioxide may be provided from any convenient source, for example, a vessel of compressed CO₂ gas or as a product of a non-interfering chemical reaction.

Without wishing to be bound by theory, the inventors suggest that it is possible that when CO₂ is added to water it reacts to form carbonic acid and bicarbonate ion (H₂CO₃ and HCO₃⁻) and, at pH values of 7 or lower, very small amounts of carbonate ion (CO₃²⁻). In the complete absence of CO₂, pure water has a pH of about 7.00. When water has been vigorously contacted with CO₂ for about 15 minutes, the pH is about 4.5. When such water of pH 4.5 is then heated at 70° C. for about 40 minutes, a substantial amount of CO₂ is expelled and the pH is about 6.75. Embodiments of the invention provide compounds that in aqueous solution exist in one state in the substantial absence of CO₂, and in another state in the presence of CO₂. In other words, the protonated form of the compound must be sufficiently acidic so that it loses its proton and protonates bicarbonate anion to a significant extent. However, if the protonated form of the compound is too acidic, its conjugate base will not be protonated by carbonic acid to a significant extent. Thus the most useful pKa range for compounds of the invention is defined.

pKa is used to quantify at what pH a proton can be abstracted from a molecule. For this reason it is associated with a protonated form of a molecule. In their protonated (uncharged or neutral) state, certain compounds of the invention have a pKa that is within about 2.5 pH units of the pKa of carbonic acid (6.4), where “carbonic acid” refers to the mixture of H₂CO₃ and dissolved CO₂ that results when CO₂ is present in water.

Embodiments of the invention include compounds with a hydrophobic moiety (e.g., hydrocarbon chain) represented by a wiggly line in equation (1), and at least one heteroatom (represented by E in equation (1)) that is a hydrogen donor in its neutral state and a hydrogen acceptor in its anionic state. In the presence of CO₂, such a compound in aqueous solution is in a neutral state and its heteroatom is protonated. In the substantial absence of CO₂, the compound in aqueous solution is in an anionic state and its heteroatom is deprotonated and negatively charged. See equation (1) below for a generic chemical equation for this reversible reaction. For clarity sodium is used as a counterion; however, as one skilled in the art will recognize, other cationic counterions could be used in place of sodium (e.g., K⁺, Li⁺, Rb⁺, Cs⁺, NH₄⁺, a transition metal ion, Hg⁺, U⁺, Cr⁺, Pb⁺, Pu⁺, NR₄⁺, NRH₃⁺, NR₂H₂⁺, or NR₃H⁺, where R is a lower alkyl). Cationic counterions that have a charge of +2 or +3 can also be used (e.g., Ca²⁺, Fe³⁺); as one skilled in the art of the invention will recognize, fewer molar equivalents of cationic counterion relative to anionic surfactant would be required for charge balance in such cases.

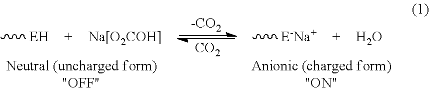

Compounds of the invention, in aqueous solution, are reversibly switchable from their anionic state to their neutral state and back again, over and over, depending on whether CO₂ is present or substantially absent. When the heteroatom is negatively charged, the portion of the compound that is charged is hydrophilic. Thus, when negatively charged, the molecule possesses both a hydrophobic moiety (e.g., a long hydrocarbon chain; a hydrocarbon chain with one or more aryl moieties in the chain; a hydrocarbon chain with one or more non-aromatic rings in the chain where the hydrocarbon chain and/or the ring moieties may be substituted or unsubstituted) and a hydrophilic moiety. By possessing both hydrophobic and hydrophilic moieties, this negatively charged compound acts as a surfactant.

In initial studies, the trigger used to expel CO₂ from solution and to switch from “OFF” (neutral) to “ON” (anionic) was heat. However, CO₂ was also shown to be expelled, and the OFF form was converted to the ON form merely by exposing the solution to air (see Example 6D). Other methods of converting OFF to ON include exposing the solution to a flushing gas either by passive exposure or active bubbling. A flushing gas can be any nonreactive gas or mixture of gases that contains insufficient CO₂ to cause the switch from anionic to neutral, e.g., a gas that contains substantially no carbon dioxide. Preferably, the gas is non-toxic. Preferred gases that are substantially free of CO₂ include, for example, argon, N₂, air that has insufficient carbon dioxide to switch the anionic form to the neutral form, air that has insufficient carbon dioxide to maintain the neutral form, and air with the carbon dioxide component removed.

In some cases, normal air, without any removal of either the existing CO₂ or the H₂O content, will suffice. Conveniently, such exposure is achieved by bubbling the gas through the mixture or by any other means of providing efficient contact between the liquid and gas phases. However, it is important to recognize that heating the mixture is an alternative method of driving off the CO₂, and this method of converting the neutral form to the anionic form is also encompassed by the invention. In certain situations, especially if speed is desired, both bubbling (or other means of providing efficient gas-liquid contact) and heat can be employed. Heat may be supplied from an external heat source, preheated nonreactive gas, exothermic dissolution of gas in the liquid phase, or an exothermic process or reaction occurring inside the liquid.

In some embodiments, simply exposing (i.e., providing a headgas above the mixture without addition of heat or actively bubbling with gas) a mixture of OFF surfactant to air or, another inert gas that has insufficient CO₂ to maintain the surfactant in its OFF state, may be sufficient to expel the CO₂. In certain embodiments this method is too slow at room temperature to be practical. However, when the mixture already exists at a sufficiently elevated temperature, this method could work adequately. In certain embodiments, loss of CO₂ is hastened by spreading such solutions into thin films and exposing the films to a flushing gas such as air. In further embodiments, loss of CO₂ is hastened by the addition of additives such as materials having high surface area which act as nucleating sites. It is also possible to trap CO₂ chemically to remove it from solution instead of physically removing it.

If desired, exposure to CO₂ is hastened by spreading such solutions into thin films and exposing to a CO₂-rich gas or atmosphere.

Depending on the pKa of the neutral form of the compound, it may be advantageous to adjust the pH of the solution to drive one direction of the reversible reaction further towards completion. However, if it is desirable to again switch the surfactant back, the amount of pH adjustment must not prevent subsequent switches. For example, where the pKa is about 6 for the neutral switchable compound, the compound may readily switch from anionic to neutral, but to obtain completion of the switch back from neutral to anionic, the presence of a base may be advantageous. The base may be added at the time of switching to the anionic form or it may already be present in the system from earlier stages in the process. In studies described herein, the addition of a base was found to work well (see Example 2), and did not interfere with the ON to OFF reaction. An added base can be chosen from inorganic bases such as, for example, NaOH, KOH, or NaHCO₃, or organic bases such as, for example, amine, amidine, guanidine, or an alkoxide salt such as, for example, sodium methoxide. Note that this added base is in addition to the base that is stoichiometrically required for the initial preparation of the “ON” form.

For embodiments of the invention where the pKa of the neutral form is quite close to the pH of an aqueous solution that is saturated with CO₂ (pH 4.5), added base would not be needed to switch from the neutral form to the anionic form. The compound may readily switch from neutral to anionic, but to obtain completion of the switch back from anionic to neutral, the presence of either (i) an additional acid, or (ii) the removal of the neutral form from the water by precipitation or by partitioning into an organic liquid, may be advantageous. If additional acid is added, it may be added at the time of switching to the neutral form or it may already be present in the system from an earlier stage in the process.

As discussed above, embodiments of the invention include compounds with a hydrophobic moiety and at least one heteroatom. Examples of hydrophobic moieties include: C₄-C₁₀₀ aliphatic moieties; non-aromatic cyclic moieties; aryl moieties; and combinations thereof. In some embodiments, the heteroatom is located at one end of the hydrophobic moiety. In certain embodiments, the hydrophobic portion has a head group at one end. For example, the headgroup may be an aryl moiety, a non-aromatic cyclic moiety, or a combination thereof. The headgroup may be substituted or unsubstituted. The heteroatom may be part of the headgroup, or may be attached to the headgroup. The headgroup may be substituted or unsubstituted. The headgroup may be substituted by the heteroatom or by a moiety including the heteroatom. Examples of headgroups that include heteroatoms include pyridines and phenols. The heteroatom could be, for example, O, S, or Se. The protonated heteroatom moiety could be, OH, SH or SeH. Thus the protonated headgroups could be, for example, alcohols, phenols, or thiols. Although not wishing to be bound by theory the inventors suggest that an aliphatic alcohol may not be efficient at reversibly switching for ON to OFF forms because most such alcohols have pKa values much higher than that of carbonic acid and because many aliphatic alcohols would probably, in the charged form, be partly or completely protonated by water. In some embodiments, the headgroup is separated from the hydrophobic moiety by a spacer moiety that it is advantageous to include in order to adjust the properties of the molecule; examples of such spacer moieties might include an aryl group, an ester group, a carbonyl group, one or more —CH₂CH₂O— groups (or substituted variations thereof), or combinations of such moieties.

An embodiment of the invention uses compounds of the invention to remediate soil that has been contaminated by a hydrophobic material (e.g., fossil fuels, oil, vegetable oils, bitumen, lubricants, etc). As described herein, it is possible to efficiently clean solid materials (e.g, rocks, sand, clay, earth, soil, fabric, machinery, plastic, rubber, metal, etc) that are coated (or permeated, that is, mixed in some manner) with a hydrophobic substance, using compounds of the invention. Advantageously, it is possible to recapture the contaminant in a substantially pure form. Working Examples 7A-H and Tables 7-9 provide experimental procedures and data regarding the efficiency of cleaning solid materials (e.g., sand) and recapturing of a hydrophobic contaminant (e.g., crude oil).

Notably, compounds of the invention were more effective (i.e., greater than 80% removal relative to 68% and 40%) at removing oil from the contaminated sand at room temperature than were conventional surfactants Triton X-100 and sodium dodecyl sulfate (SDS). At 50° C., compounds of the invention were comparably effective relative to these conventional surfactants.

Importantly, recapture of the crude oil was possible with the compounds of the invention after exposure to CO₂, whereas recapture of oil using traditional surfactants is economically unfeasible. As described herein, switchable anionic surfactants remove oil as well or better than traditional surfactants, work at a lower temperature than traditional surfactants, and are switchable, thereby allowing hydrophobic components (e.g., oil) to be separated from the aqueous wash solution when the cleaning process is done.

After cleaning (solubilizing a contaminant) using a compound of the invention in its anionic surfactant form, it is possible recapture the contaminant in a substantially pure form by converting the compound of the invention to its neutral non-surfactant form. Thus, soil that is contaminated by oil can be readily converted to (1) substantially clean soil, (2) substantially pure oil, and (3) reusable aqueous solution or aqueous mixture of water and a compound of the invention.

The invention also provides a method for separating two immiscible liquids using a reversibly switchable surfactant. The invention further provides a method for maintaining an emulsion using a reversibly switchable surfactant. The surfactant may then be turned OFF and the immiscible liquids separated.

In certain embodiments of the invention, two immiscible liquids are (1) water or an aqueous solution and (2) a water-immiscible liquid such as a solvent, a reagent, a monomer, an oil, a hydrocarbon, a halocarbon, or a hydrohalocarbon. The water-immiscible liquid could be pure or a mixture. Solvents include, for example and without limitation, alkanes, ethers, amines, esters, aromatics, higher alcohols, and combinations thereof. Monomers include, for example and without limitation, styrene, chloroprene, butadiene, acrylonitrile, tetrafluoroethylene, methylmethacrylate, vinylacetate, isoprene, and combinations thereof. Oils include, for example and without limitation, crude oil, bitumen, refined mineral oils, vegetable oils, seed oils (such as soybean oil and canola oil), fish and whale oils, animal-derived oils, and combinations thereof. Halocarbons include, for example and without limitation,(trifluoromethyl)benzene, chlorobenzene, chloroform, chlorodibromomethane, partially fluorinated alkanes, and combinations thereof. A water-immiscible liquid could be a gas at standard temperature and pressure but a liquid or supercritical fluid at the conditions of the application. (Supercritical fluids, while not technically liquids, are intended to be included when liquids are discussed.) In other embodiments of the invention, two immiscible liquids are a more polar liquid and a less polar liquid. Polar compounds have more hydrogen bonding and/or greater dipole moments and/or charge separation. They include, for example, solvents, reagents and monomers such as alcohols (e.g., methanol, ethylene glycol, glycerol, vinyl alcohols), carboxylic acids (e.g., acrylic acid, methacrylic acid, acetic acid, maleic acid), nitrites (e.g., acetonitrile), amides (e.g., acrylamide, dimethylformamide), sulfoxides (e.g., dimethylsulfoxide), carbonates (e.g., propyl carbonate), sulfones (e.g., dimethylsulfone), ionic liquids, and other highly polar liquids, e.g., hexamethylphosphorus triamide, nitromethane, 1-methylpyrrolidin-2-one, sulfolane, and tetramethylurea. Less polar compounds have less hydrogen bonding and/or lesser dipole moments and/or less charge separation. Less polar liquids include solvents, reagents, monomers, oils, hydrocarbons, halocarbons, and hydrohalocarbons as described previously. These could be pure liquids, mixtures or solutions.

In other embodiments of the invention, two immiscible liquids are two immiscible aqueous solutions, for example, an aqueous solution of polyethylene glycol and an aqueous solution of a salt.

The switchable surfactant system according to the invention can facilitate water/solid separations in mining. In mineral recovery, switchable surfactants may be suitable as flotation reagents, which are mineral-specific agents that adsorb to the mineral particles to render them hydrophobic and therefore likely to float upon aeration. Flotation reagents designed on the basis of switchable surfactants could be readily removed from minerals and recycled.

The switchable surfactant system of the invention can be employed for extraction of a hydrophobic substance from a mixture or matrix using a combination of water or aqueous solution and surfactant, for example, oil from porous rock, spilled oil from contaminated soil, desirable organic compounds from biological material (plant or animal), ink from paper, dirt from clothing. Analogously, the invention provides a method for extracting a hydrophilic substance from a mixture or matrix using a combination of organic solvent and surfactant, for example, caffeine from coffee, metal salts from soil, salts or polyols (e.g., sugars) from organic mixtures. In each case, the extracted substance can be recovered from solvent by turning OFF the switchable surfactant.

Switchable surfactants of the invention can be useful in oil-sands separation processes, cleaning of beaches and wetlands after oil-spills, and in cleaning of equipment. Reversibly switching between surfactant and non-surfactant has particular utility for the oil industry.

Reversibly switchable surfactants can be useful additives in polymerization reactions. A switchable surfactant can be used in an emulsion or microsuspension polymerization of water insoluble polymers. This permits manufacture of very high molecular weight polymers which are recovered from solution by switching off the surfactant, filtering and drying the obtained solid. In general, such high molecular weight polymers are difficult to produce in a solution polymerization process without surfactants because of their tendency to form gels. Switchable surfactants of the invention could protect surfaces of nanoparticles, colloids, latexes, and other particulates during synthesis and use. In the absence of a coating of surfactant, such particles tend to agglomerate. But, in many cases, once the synthesis is complete, the presence of surfactant is no longer desirable. For example, in preparation of supported metal catalysts, complete removal of surfactant is desired, but it is difficult with non-switchable surfactants, since they bind strongly to the surface.

It should also be noted that switchable surfactants of the invention have application in latex paints and other coating formulations since they can turn OFF when the paint or coating is applied to a surface in air. Whether deactivation of the surfactant is desired, or its complete removal, switchable surfactants, of the invention offer advantages. Their presence would allow the desired polymer particle size to be achieved while allowing the polymer to precipitate from solution when the switchable surfactant is turned OFF.

A switchable surfactant of the invention can be used in inverse emulsion polymerization of water soluble polymers. In general, water-soluble polymers and/or hygroscopic polymers are prepared by polymerization of an inverse emulsion of a monomer in a hydrophobic solvent. An inverse emulsion has as its continuous phase an organic solvent and has micelle cores present to surround a hydrophilic monomer. With the presence of a switchable surfactant, this inverse emulsion mixture is stabilized and a polymerization reaction is possible. At completion of the polymerization, the surfactant is switched off by application of flushing gas to the mixture. The OFF surfactant then partitions into the organic solvent and the polymer precipitates. This permits manufacture of very high molecular weight polymers which are recovered from the inverse emulsion and dried to produce a product (dry-form high MW or branched polymers) that could not be achieved in a standard solution polymerization processes because of the tendency for such products to form gels. Low HLB (hydrophile/lipophile balance) switchable surfactants are preferred in this application, and the surfactant should not act as a chain-transfer agent. Polymers that are expected to be readily prepared by this method include, for example, polyacrylamide, polyacrylic acid, polymethacrylic acid, alkali metal salts of polyacrylic acid or polymethacrylic acid, tetraalkylammonium salts of polyacrylic acid or polymethacrylic acid, polyvinylalcohols, and other hygroscopic polymers or polymers that are substantially soluble in water or that swell in water.

Another application for reversibly switchable surfactants is protection and deprotection of nanoparticles. Nanoparticles and other materials are frequently temporarily protected during synthetic procedures by traditional surfactants. They could be more readily deprotected and cleaned if reversibly switchable surfactants were used.

The switchable surfactants and methods of use thereof according to the invention can lessen environmental impact of industrial processes, both by saving energy normally expended during separations and by improving the purity of wastewater emitted from production facilities. The presence of a switchable surfactant in waste effluent could lead to significantly less environmental damage, since effluent can be readily decontaminated by treatment with the appropriate trigger prior to its release into the environment.

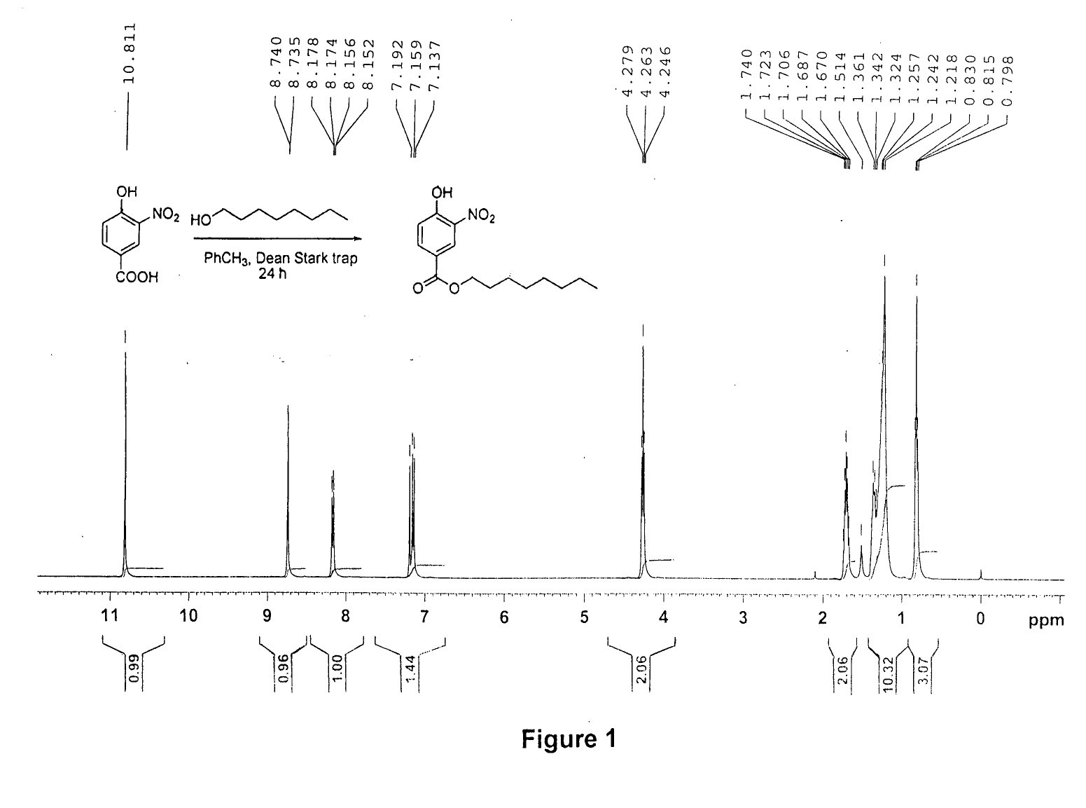

An example of a switchable anionic surfactant is n-octyl 4-hydroxy-3-nitrobenzoate (1N), shown below. Compound (1N) was synthesized and characterized as described in Example 1 and shown in FIG. 1. Surface activity was studied as described in Example 3 for compound (1A), which is the ON form of the surfactant, and for compound (1N), which is the OFF form.

Compound (1N) has a pKa of approximately 5.5. As discussed previously, when placed in aqueous solution or in contact with aqueous solution, compound (1N) may remain protonated depending on the pH of the aqueous solution. However, if the pH of the solution is substantially above 5.5, most of this compound would be in its deprotonated anionic form (1A) (see equations (2) and (3)). This anionic form (1A) has significant surface activity.

When compound (1A) is placed in aqueous solution or in contact with aqueous solution, compound (1A) may remain deprotonated or may become protonated depending on the pH of the aqueous solution. If the pH of the solution is substantially below 5.5, most of this compound would be in its protonated neutral form (1N). The neutral form has less surface activity.

When compound (1A) is desired in an aqueous solution, it can either be added directly, or it can be formed in situ by adding compound (1N) and converting (1N) to compound (1A). When compound (1N), the OFF form, is placed in solution, it can be turned ON by treating it with a base. When it has yet not been through an ON-OFF cycle, there may be little to no CO₂ in the aqueous solution. Accordingly it may require addition of a base to switch ON. Once the solution includes dissolved CO₂, then the OFF form can be switched ON by depleting the solution of CO₂. Such depletion of CO₂ is possible by: heating; exposing the solution to flushing gas; exposing the solution to air; exposing the solution to a gas or gases that has insufficient CO₂ content to convert the ON state to the OFF state; flushing with a gas or gases that has insufficient CO₂ content to convert the ON state to the OFF state; or a combination thereof.

Studies that are described herein showed that the compound present in solution when compound (1N) and Na₂CO₃ (8 mole equivalents) are dissolved together in deuterated water is the anionic form (1A). Specifically, its ¹H NMR spectrum matched that obtained by dissolving the sodium salt of compound (1A) in deuterated water. Studies that are described herein also show that the organic compound that is precipitated out of a solution of (1A) and Na₂CO₃ in water in the presence of CO₂ is compound (1N)—the neutral state. Specifically, the precipitate, when dissolved in CDCl₃, had a ¹H NMR spectrum that was identical to that of compound (1N) dissolved in CDCl₃.

Variations to the structure of compounds (1N) and (1A) are well within the skill of the person of ordinary skill in the art pertaining to the invention. These include minor substitutions, varying the length of the hydrocarbon chain of the ester, and the like.

To switch from the anionic state (ON) to the neutral state (OFF), CO₂ was bubbled through the solution. To switch from the neutral state (OFF) to the anionic state (ON), CO₂ was removed by heating the solution in the presence of added base to help drive the reaction to completion (see equations (2), (3), and (4)). Without wishing to be bound by theory, the inventors suggest that the reason that added base was needed to drive the reaction to completion is because the pKa of this compound (5.5) is quite close to the pH of an aqueous solution with CO₂ substantially removed (pH ˜6).

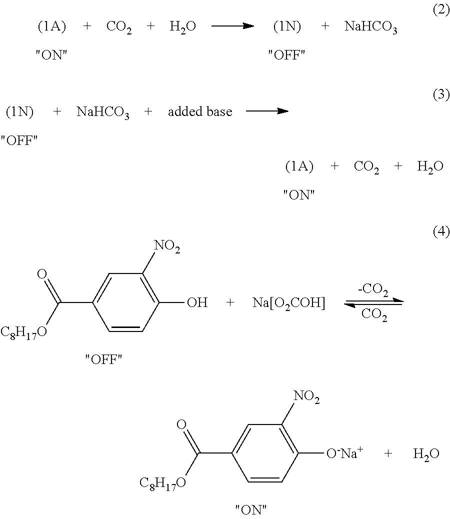

### WORKING EXAMPLES

### Materials and Methods

CO₂ (Praxair, SFC grade, 99.998%), argon (Praxair, 99.998%) and air (Praxair, extra-dry grade) were used as received. Unless otherwise noted, reagents were received from Aldrich (Oakville, Ontario, Canada).

To turn a surfactant OFF, CO₂ gas was slowly bubbled for 5 min through the solution. To turn a surfactant ON, the solutions was heated to 70° C. for 40 min by immersing the reaction flask in an oil bath that was held at a constant temperature of 70° C. Unless otherwise specified, all vials were tightly capped using a screw cap, the gap between tightened cap and Vial was covered with PARAFILM™ to ensure that there was no leakage nor displacement of gases.

In certain examples herein, when the added base was NaHCO₃, instead of adding NaHCO₃ to the solution directly, it was formed in situ by adding Na₂CO₃ to the aqueous solution, which Na₂CO₃ in the presence of CO₂ converts to NaHCO₃.

Unless otherwise specified, water that was used in studies described herein was municipal tap water from Kingston, Ontario, Canada that was deionized by reverse osmosis and then piped through a MilliQ Synthesis A10 apparatus (Millipore SAS, Molsheim, France) for further purification.

### Example 1

### Synthesis of n-octyl 4-hydroxy-3-nitrobenzoate (1N)

A mixture of 4-hydroxy-3-nitrobenzoic acid (5.00 g, 27.32 mmol), n-octanol (7.1 g, 8.62 mL, 54.64 mmol), para-toluenesutfonic acid (0.050 g) and toluene (200 mL) were refluxed for 24 h under a Dean-Stark trap to remove water by azeotropic distillation. After completion of the reaction, toluene was evaporated under reduced pressure. The residue was diluted with wet diethyl ether (400 mL) and a solution of potassium hydroxide (2.00 g, 35 mmol) in ethanol (95%, 40 mL) was added. A dark orange solid was obtained. The solid was filtered through a scintered glass filter, washed with diethyl ether (2×20 mL) and dried by exposure to air. The crude product was added to a cold aqueous solution (200 mL of water) that was then gradually acidified with concentrated hydrochloric acid until the solution was acidic (by pH paper). An oily liquid formed that solidified when cooled (n-octyl 4-hydroxy-3-nitrobenzoate, (1N)). Yield 87%. UV: λₘₐₓ=240 nm, εₘₐₓ=57412 dm³ mole⁻¹cm⁻¹ and another significant peak at λ=336 nm, ε=6265 dm³ mole⁻¹cm⁻¹. IR (KBr): 3270, 2961, 2922, 2858, 1717, 1631, 1582, 1490, 1477, 1426, 1331, 1282, 1178, 1147, 1037, 1018, 971, 928, 864, 834, 761, 695, 636 cm⁻¹. ¹H NMR (CDCl₃, 400 MHz) (see FIG. 1): δ 0.80-0.83 (t, J=6.4 Hz, 3H), 1.22-1.36 (m, 10H), 1.67-1.74 (m, 2H), 4.25-4.28 (t, J=6.6 Hz, 2H), 7.14-7.16 (d, J=8.8 Hz, 1H), 8.15-8.18 (dd, J=1.6 , 8.8 Hz, 1H), 8.73-8.74 (d, J=2 Hz, 1H), 10.81 (s, 1H). ¹³C NMR (DMSO d₅, 100 MHz): δ 13.83, 22.08, 25.45, 28.13, 28.61, 28.66, 31.23, 64.88, 119.36, 120.63, 126.58, 135.11, 136.69, 155.87, 163.99.

### Example 2

### Switching Efficiency of Compounds (1A) and (1N) as Determined by ¹H NMR Spectroscopic Studies

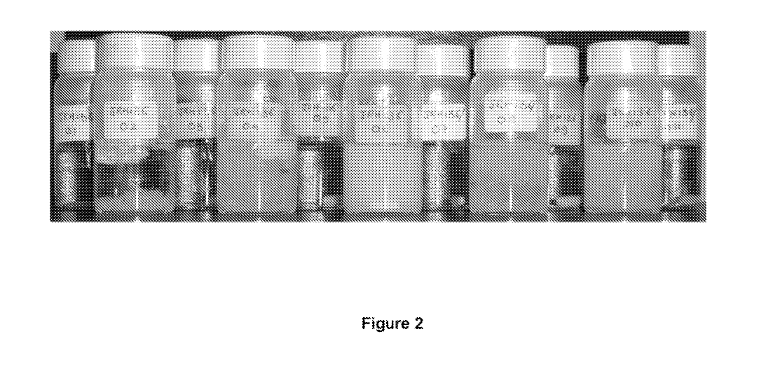

The following study confirmed that compounds (1A) and (1N) reversibly convert from the neutral form (1N) to the anionic form (1A) and back to the neutral form over and over again with substantially no loss of product. The conversion was verified visually since the neutral form was not soluble in aqueous solution and appeared as a white precipitate in colourless liquid while the anionic form was fully soluble in aqueous solution and appeared as a clear yellow liquid (see FIG. 2). In addition to visual examination, ¹H NMR studies that are described below confirmed the presence of the hydroxyl proton in the spectra of the neutral form.

Two methods were used for demonstrating the efficiency of the switching process, method A using [(CH₃)₄N]I and method B using dimethylformamide (DMF) as internal standards. In both methods, to quantify the concentration of surfactant in solution, its ¹H NMR integration was compared to one of two internal standards of known concentration. In addition to the difference in which internal standard was used, the two methods can also be distinguished from each other on the basis of the timing of the addition of the internal standard. In method A, the standard remains in the solution during the process of switching. In method B, the switching is executed first and the DMF solution of known strength and quantity is added in to the solution.

In both methods, the surfactant was turned OFF with CO₂ by positioning the needle that supplied the CO₂ to the surfactant solution above the solution in such a way that the slow flow of CO₂ was allowed to interact with the surfactant solution at the surface. The switching from ON to OFF required 5 min or less. Increasing the surface area, for example, by slightly tilting the vial, expedited the switching from ON to OFF. To turn the surfactant ON, the solution was stirred at 70° C. for 40 min as described above. After each ON cycle, 0.5 mL of D₂O was added to the solution to compensate for the loss in the volume caused by evaporation.

### Example 2A

### Method A

The surfactant solution was prepared by combining 0.046 g of octyl 4-hydroxy-3-nitrobenzoate (1N) and 0.136 g of Na₂CO₃ in 16.0 mL of D₂O. To this solution, 11.5 mg of [(CH₃)₄N]I was added. The resultant solution was homogenized by sonication for 2 min. 1.00 mL of this solution was transferred using a Hamilton syringe, which had a least count value of 0.005 mL into eleven different vials. Stirring bars were introduced in vials 3 to 11. Vial 1 was the control solution, so it did not enter into the ON/OFF cycle. The surfactant solutions were turned OFF and ON by the procedure described in Example 3.

As the standard, [(CH₃)₄N]I was added early into the surfactant solution. All the solutions were filtered and the ¹H NMR spectra of all these solutions were recorded. In all cases, the integration of [(CH₃)₄N]I was set to 100 and compared with the integration of aromatic proton at δ ˜7.3. The vials in which the ON/OFF cycles concluded with the CO₂ bubbling to turn the surfactant OFF (i.e., vials 2, 4, 6, 8 and 10), did not show any detectable signal for the surfactant. Thus the concentration of the surfactant in these solutions was substantially zero. The integration values of the protons at δ ˜7.3 (see Table 2) indicated that in all vials in which the cycle concluded with turning the surfactant ON (i.e., vials 3, 5, 7, 9, and 11) had the same surfactant strength as vial 1. Thus the experimental results established that the surfactant turns ON and OFF with substantially 100% efficiency.

### Example 2B

### Method B

The surfactant solution was prepared by combining 0.046 g of octyl 4-hydroxy-3-nitrobenzoate and 0.136 g of Na₂CO₃ in 16.0 mL of D₂O. The resultant solution was homogenized by sonication for 2 min. 1.00 mL of this solution was transferred using a Hamilton syringe into thirteen different vials (1, 2, 2*, 3, 4, 5, 6, 7, 8, 9, 10, 11 and 12). Stirring bars were introduced in vials 2* through 12. Vial 1 was the control solution, so it did not enter into the ON/OFF cycle. The surfactant in vial 2 was turned OFF by the procedure described above. The solution in vial 2* was turned OFF with CO₂ and was then left stirring uncapped at room temperature for 18 h. Details of the treatment of the individual vials are presented in Table 1. All the vials (excluding 2*) were tightly capped and left at room temperature for 18 h to confirm the stability of the systems in both the ON/OFF forms upon standing. The solution in vial 2* turned yellow and clear after 18 h stirring at room temperature, indicating that perhaps the compound (1N) that separated from the solution on CO₂ treatment, slowly dissolved again into the solution upon stirring at room temperature.

The standard solution of DMF was prepared by mixing 0.005 mL of DMF in 8.0 mL of D₂O. 0.300 mL of the standard solution was added to all vials. The solutions in all vials was homogenized well and filtered. The ¹H NMR spectra of all solutions were recorded. In all cases, the integration of (CH₃)₂NCHO was set to 100 and compared with the integration of the aromatic proton at ˜7.3 δ (Table 3). The solutions in each vial were turned OFF and ON by methods described above and by the cycles shown in Table 1. Vial 1 was the control solution, so it did not enter into an ON/OFF cycle. In vial 2 and vial 2*, the surfactant was turned OFF. In vial 2*, following the OFF-turning, the uncapped vial contents were stirred at room temperature for 48 h. The vials in which the ON/OFF cycle concluded with the CO₂ bubbling to turn the surfactant OFF (vials, 2, 4, 6, 8, 10 and 12), did not show a detectable signal for the surfactant, i.e. zero concentrations of the surfactant in the solution. NMR proton integration values at δ ˜7.3, indicate that within an acceptable error, in all the vials in which the cycle concluded with the surfactant ON (vials 3, 5, 7, 9 and 11) had the same surfactant concentration as vial 1. It is noted that the proton integration at δ ˜7.3 in the solutions of vials 2* and 3 were a perfect match (see Table 3), proving that the water insoluble compound (1N) slowly reacted with the aqueous base and was converted into compound (1A) upon stirring at room temperature. This experiment confirms that the surfactant turns ON and OFF with substantially 100% efficiency.

### Example 3

### Surface Tension Studies

### Example 3A

### Preparation of Solutions for the Surface Tension Measurements

A stock solution of compound (1A) was prepared by combining 0.460 g (1.559 mmol) of octyl 4-hydroxy-3-nitrobenzoate (1N) and 1.36 g (12.83 mmol) of Na₂CO₃ in 160 mL of deionized H₂O. The resultant solution was homogenized by sonication for 2 min using a sonicating machine (model Semiautomatic Model 21 Tensiomat, available from Fisher Scientific, Ottawa, Canada). 10 mL of the homogeneous solution was transferred into each of thirteen vials (13×10 mL, vial numbers: 1, 2, 2*, 3, 4, 5, 6, 7, 8, 9, 10, 11 and 12). The levels of solution in all the vials were marked using a marker pen on the outside of the vials. A stir bar was introduced in vials 2* through 12.

### Example 3B

### Influence of Switchability of octyl 4-hydroxy-3-nitrobenzoate (1N) on the Surface Tension of a Solution

The solutions in each vial were turned OFF and back ON according to Table 1. Vial 1 was the control solution, so it did not enter into an ON/OFF cycle. In vials 2 and 2*, the surfactant was turned OFF. In vial 2*, following the OFF-turning, the uncapped vial contents were stirred at room temperature for 48 h.

Vials (other than vial 2*) were left at room temperature for 18 h prior to surface tension measurements. Where necessary, water was added to the vials until the total volume matched the original marked levels.

The contents of vials with even numbers (OFF) were filtered by passing them through a Kimwipe™ plug in a pipette to obtain a clear solution. All the odd numbered vials were visibly free of solids and did not require filtration.

The solution in vial 2* slowly turned yellow and over time it became clearer and an enhancement in the intensity of its colour was seen. Without wishing to be bound by theory, the inventors suggest that (1N) that precipitated from solution upon CO₂ treatment, slowly dissolved into the solution upon stirring at room temperature and was converted to the sodium salt of (1A). As the solution was stirred at room temperature, the (1N)-precipitate slowly dissolved and as the (1A) formed the solution became more and more yellow. At 48 h, the solution's clarity and colour intensity were at their maximum and the solution was transparent and had its strongest intensity.

### Example 3C

### Tensiometer Measurements

Surface tension for all solutions was measured in triplicate. The three values of surface tension for each solution remained within ±1 dynes/cm indicating good reproducibility. The tensiometer (Surface Tensiomat 21, Model 14814, available from Fischer Scientific, Ottawa, Canada) was calibrated using deionized water. The initial value of 72 dynes/cm obtained for water was acceptable within experimental error. For each solution, the average value of the three measurements was obtained and is presented in Table 4. Following all measurements, the surface tension of water was recorded again and was found to be 71 dynes/cm.

In the ON form (vials 1, 3, 5, 7, 9, and 11) the average value of the surface tension was 34 dynes/cm and in the OFF form (vials 2, 4, 6, 8, 10 and 12) the average value was 61 dynes/cm (see Table 4). For the vials in which the ON/OFF cycle concluded with the surfactant in the ON form, the surface tension remained very close (±1 dyne/cm) to its average value (34 dynes/cm), but for the vials in which the ON/OFF cycle concluded with the surfactant in the OFF form, the surface tension values varied by ±5 dyne/cm (see Table 4) with the highest being 66 and the lowest being 57 dyne/cm.

### Example 4

### pH Studies

For pH studies described below, a pH meter (Orion 4 Star Conductivity Benchtop, available from ThermoElectron Corporation, Mass., USA) was used. The pH meter was calibrated before the measurements as per the instructions provided with the pH meter. All solutions were prepared using deionized water. All pH measurements were done at 25° C.

Surfactant solution was prepared by dissolving n-octyl 4-hydroxy-3-nitrobenzoate (0.443 g, 1.502 mmol) and anhydrous Na₂CO₃ (1.27 g, 12.02 mmol) in 150 mL of deionized water. The resultant solution was homogenized by sonication for 2 min. Aliquots (10.0 mL) of this solution were transferred into eleven different vials numbered as 1, 2, 3, 4, 5, 6, 7, 8, 9, 10, and 11 by Hamilton syringe. The level of the solution in each vial was marked using a marker. Stirring bars were introduced in vials 3 to 11. Vial 1 was a control solution, so it did not enter into the ON/OFF cycle. To turn the surfactant OFF, CO₂ was slowly bubbled through a solution for 10 min (formation of the OFF form was observed by formation of light yellow solid precipitate). To turn the surfactant ON the solution was heated and stirred while immersed in an oil bath preset at 70° C., for 40 min. The solution in vial 2 was turned OFF. The solution in vial 3 was first turned OFF and then ON. The solution in vial 4 was first turned OFF then ON and finally OFF. The details of the treatment given to each vial is summarized in Table 1. After completion of the indicated cycle, each vial was first allowed to cool down to room temperature (if the cycle marked its completion with the surfactant in ON form), the solution was then diluted with deionized water. To be consistent with the previous experiments, all the vials were tightly capped (using PARAFILM™) and allowed to stand for 18 h at room temperature (see FIG. 2).

As can be seen from the results (Table 6), the average pH value of the solution in ON form (1A) is 9.31 and of the solutions in the OFF form (1N) is 7.73, the difference in the two average values being 1.58. The pH change is highest at 3.50 units when the solution is turned OFF for the first time. This observation can be explained by the conversion of Na₂CO₃ to NaHCO₃, when CO₂ is bubbled through the vial to turn it OFF for the first time. In the subsequent cycles, the change in pH averages 1.58 units, which is governed by the presence or absence of phenolate ions in the aqueous solution. These observations confirmed that NaHCO₃ was a good replacement to Na₂CO₃ in the preparation of the phenolate solution.

In a control experiment, the pH was measured for a 0.08 M Na₂CO₃ solution before (pH 11.42) and after (pH 6.78) bubbling with CO₂ for 15 min. This decrease in pH is likely due to the formation of both NaHCO₃ and H₂CO₃. When this solution was heated at 70° C. for 40 minutes the pH increased to 8.98.

In another control experiment, the pH of deionized water was determined at various levels of CO₂. The initial pH of 7.00 was reduced to 4.50 after 15 min of bubbling with CO₂. This pH decrease is due to the formation of H₂CO₃. When this solution was heated at 70° C. for 40 min, the pH increased to 6.75. This increase is due to decomposition of H₂CO₃ and loss of CO₂. The results of these studies are presented in Table 5.

### Example 5

### Switchability using DMSO-d₆

Since n-octyl 4-hydroxy-3-nitrobenzoate (1N) and the potassium salt of (1A) are soluble in DMSO and have some characteristic signals in the ¹H NMR spectrum, DMSO-d₈ was used as a solvent to monitor the non-aqueous switchability by NMR spectroscopy. When CO₂ was bubbled through the 0.01 M solution of potassium 4-hydroxy-3-nitrobenzoate in DMSO-d₆, no conversion was apparent by the ¹H NMR spectroscopy. Thus the switching did not occur in DMSO-d₆.

### Example 6

### Switchability Studies of Compound (1) with Triggers Na₂CO₃, CO₂ and Air

### Example 6A

### Preparing and Isolating Sodium 2-nitro-4-(octyloxycarbonyl)phenolate

Sodium 2-nitro-4-(octyloxycarbonyl)phenolate was prepared by dissolving compound (1N) in 0.1 M solution of NaOH with stirring. The addition of (1N) was continued until it does not dissolve in the aqueous alkali solution anymore. The solution was filtered and allowed to evaporate naturally. Upon evaporation, the sodium salt was obtained as a yellow solid. ¹H NMR (D₂O, 400 MHz) δ: 0.75-0.78 (t, J=6.4 Hz, 3H), 1.15 (bs, 10H), 1.47 (bs, 2H), 3.85 (bs, 2H), 6.11-6.14 (d, J=8.8 Hz, 1H), 7.07-7.09 (d, J=8.8 Hz, 1H), 7.81 (s, 1H).

### Example 6B

### Switching from Compound (1N) to Compound (1A) using Na₂CO₃

The surfactant solution of (1A) was prepared by combining 0.023 g of octyl 4-hydroxy-3-nitrobenzoate (1N) and 0.068 g of Na₂CO₃ in 8.0 mL of D₂O. The resultant solution was homogenized by sonication for 2 min and the ¹H NMR spectrum was recorded. The ¹H NMR spectrum matched that of the isolated sample of sodium 2-nitro-4-(octyloxycarbonyl)phenolate described in Example 6A.

### Example 6C

### Switching Compound (1A) to Compound (1N) using CO₂

2.0 mL of the surfactant solution of (1A) from Example 6B was transferred in a vial. CO₂ gas was slowly bubbled through this solution for 10 min. A light yellow solid separated as a precipitate. The solution was filtered and the precipitate was dried. The ¹H NMR spectrum of this precipitate matched the ¹H NMR spectrum of octyl 4-hydroxy-3-nitrobenzoate, compound (1N). ¹H NMR (CDCl₃, 400 MHz) δ: 0.80-0.83 (t, J=6.4 Hz, 3H), 1.22-1.36 (m, 10H), 1.67-1.74 (m, 2H), 4.25-4.28 (t, J=6.6 Hz, 2H), 7.14-7.16 (d, J=8.8 Hz, 1H), 8.15-8.18 (dd, J=1.6, 8.8 Hz, 1H), 8.73-8.74 (d, J=2 Hz, 1H), 10.81 (s, 1H).

### Example 6B

### Switching from Compound (1N) to Compound (1A) using Exposure to Air

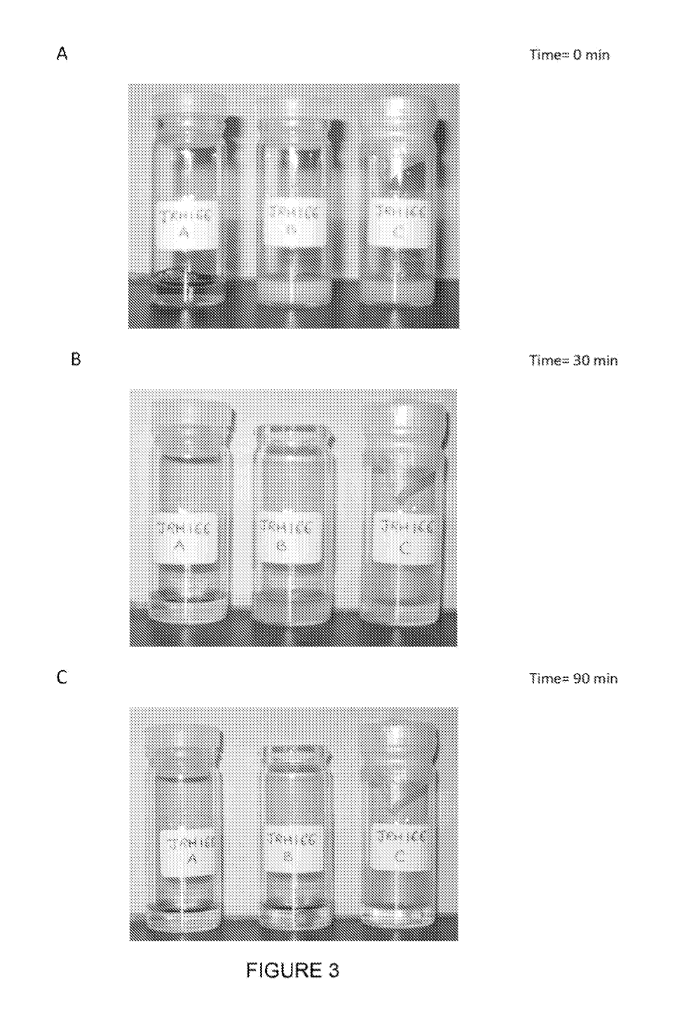

A solution of anionic surfactant (1A) was prepared by combining octyl 4-hydroxy-3-nitrobenzoate (1N) (0.023 g) and Na₂CO₃ (0.068 g) in 8.0 mL of D₂O. The resultant solution was homogenized by sonication for 2 min. Aliquots (1.00 mL) of this solution were transferred by Hamilton syringe with a least count of 0.005 mL, into three different vials, which were labeled as vials A, B and C. Visual observations were made of vials A, B and C after 0, 30 and 90 min of stirring at room temperature under separate conditions as described below and are presented in FIG. 3A, 3B and 3C, respectively. Vial A was the control solution, so it did not enter into the ON/OFF cycle. The solution in vial A remained in the ON form (1A) throughout the experiment and it appeared as a yellow homogeneous solution that was free of any solids. A magnetic stir bar was introduced in vials B and C. The surfactant solutions in vials B and C were turned OFF by allowing a stream of CO₂ provided by a needle to blow, CO₂ at the surface of the solutions (the needle was not allowed to dip or touch the surface of the solution). Vial C was then capped and its cap was supplemented by PARAFILM™ to inhibit CO₂ leakage. Thus at the beginning of this experiment, which is pictured in FIG. 3A, vial A held a solution in the ON form, and vials B and C held a solution in the OFF form. Vial B was then uncapped and stirred under passive exposure to air. Vial C was stirred under a passive CO₂ headgas.

Vial C held a colourless liquid with white precipitate present. The white precipitate was visible throughout the experiment (note that in FIG. 3B and FIG. 3C vial C's precipitate is difficult to see in the photographs since it had settled onto the bottom of the vial). After 90 minutes of stirring (See FIG. 3C) vial C still held compound (1N), which was confirmed by ¹H NMR. The solution in vial C was slightly yellow after 90 minutes of stirring. The small amount of compound (1A) that would be responsible for this subtle colour change is explained by seepage of air into the vial and solution since the CO₂ headgas was not actively maintained.

Importantly, as shown in FIG. 3B the solution in vial B after 30 minutes of stirring under air exposure had changed and appeared yellow was some solid visible in the solution. After 90 minutes of stirring (see FIG. 3C), vial B appeared as a yellow solution that was free of solids. The presence of compound (1A) in vial B and absence of compound (1N) was confirmed by ¹H NMR. In the ON form the surfactant imparts an intense yellow color to the solution.

These results prove that passive exposure to air is sufficient for converting compound (1N), which is the neutral OFF form, to compound (1A), which is the anionic ON form.

### Example 7

### Soil Washing Studies

### Example 7A

### Preparation of Artificially Contaminated Soil for Soil Washing Studies

Oil-contaminated soil was prepared in the following manner. North Sea crude oil (2.4 to 2.6 g) (Chevron, San Ramon, Calif., USA) was poured into a beaker. Tetrahydrofuran (THF) (to dissolution, approximately 50 mL) was added and the beaker and its contents were swirled to dissolve the oil. Ottawa sand (60 g) (EMD Chemicals, Gibbstown N.J., USA) was poured into the beaker and the contents of the beaker was swirled again. The beaker was left uncovered overnight at room temperature to allow the THF to evaporate. Then, the sand was broken up and mixed in the beaker. The beaker was left uncovered for a further time period so that total elapsed time for evaporation was 24 hours. The contaminated sand was then poured into a pre-weighed petrie dish, which was then placed into an oven at 110° C. and heated for 24 hours. After the petrie dish was removed from the oven and had cooled to room temperature, the sample and dish were weighed together. The contaminated sand was then transferred to a 100 mL amber vial for storage. This heat treatment was intended to simulate weathering of the contaminated soil. This procedure provided a sample of soil with 3.0 wt % oil contamination.

### Example 7B

### Preparation of Surfactant Solution at Room Temperature

A 0.5 wt % surfactant solution was prepared in the following manner: surfactant (0.25 g) was added to a 50 mL volumetric flask. Deionized water at room temperature was added to achieve a total mass of 50 g. Sonication of the solution assisted with dissolving the surfactant.

### Example 7C

### Preparation of Surfactant Solution at 50° C.

A 0.5 wt % surfactant solution was prepared in the following manner: Deionized water was heated to 50° C. Surfactant (0.25 g) was added to a 50 mL volumetric flask. The heated water was added to achieve a total mass of 50 g. Swirling and shaking of the flask was sufficient to dissolve the surfactant.

A 0.1 wt % surfactant solution was also prepared using the above-described procedure wherein 0.0583 g of surfactant was used.

### Example 7D

### Sand Washing at Room Temperature

The contaminated sand of Example 7A was washed in the following manner. Three 20 mL vials were labelled and their empty masses were, recorded. Contaminated sand (5.1 g) was added to each vial and the vial's new masses were recorded. The surfactant solution was added to each vial using a syringe to achieve 10 g of solution. A stir bar was also added. The vials were capped with TEFLON®-lined lids and shaken manually for 20 seconds. The vials were placed in a beaker, to keep them upright. The beaker was placed on a stir plate and the vials were stirred at 470 rpm for 1 hour. Each vial was then manually shaken for 20 seconds. Then the stirring was continued for a second hour. The liquid portion of the samples was poured into separate 20 mL vials and is referred to below as “first decant solution”. The original vials (and their remaining contents) were each rinsed with 50 mL of deionized water and that rinse water was then added to 50 mL Wheaton jars and is referred to below as “wash solution”.

### Example 7E

### Heated Sand Washings

Sand washing was also carried out at elevated temperature by following the procedure as described in Example 7D with the difference that vials were placed into a 50° C. water bath. The water bath was placed on a stirring hot plate and was maintained 50° C. and the vials were stirred at 470 rpm for 1 hour. Each vial was then manually shaken for 20 seconds. Then the stirring was continued for a second hour. The liquid portion of the samples was poured into separate 20 mL vials and is referred to below as “first decant solution”. The original vials were each rinsed with 50 mL of deionized water and that rinse water was then added to 50 mL Wheaton jars and is referred to below as “wash solution”.

### Example 7F

### Quantification of Oil Remaining on Washed Contaminated Sand

Oil remaining on the soil of Examples 7A-E was quantified in the following manner. An extraction was performed using 10 mL of a 1:1 dichloromethane:hexanes liquid mixture. This mixture was added to vials housing washed sand (the term “washed sand” refers to 5.1 g of contaminated sand from Example 7A that has been washed and rinsed using the procedures of Example 7D or 7E). The contents of the vials were stirred at 470 rpm for 20 minutes. After stirring, a liquid phase was removed by pipette and was filtered to trap any sand particles, by passing it through a separate pipette with a glass wool plug in its tapered section. The liquid was then collected in a pre-weighed 100 mL roundbottom flask. Another extraction with 1:1 dichloromethane:hexanes was performed, with the washings again being added to the same roundbottom flask. Three subsequent extractions were done similarly except that they were stirred for only 10 minutes. Extractions from an individual vial of sand were combined in the same roundbottom flask. The dichloromethane and hexanes were then removed from each flask by rotary evaporation. The roundbottom flask was then weighed and the difference in mass was the mass of oil that remained on the soil. Results from this procedure are presented in accompanying Table 7. Notably, compounds of the invention were more effective (i.e., greater than 80% removal relative to 68% and 40%) at removing oil from the contaminated sand at room temperature than were conventional surfactants (e.g., Triton X-100 and sodium dodecyl sulfate (SDS)). At 50° C., compounds of the invention were comparably effective relative to conventional surfactants.

### Example 7G

### Determination of Amount of Residual Oil in Aqueous Washing Solution

The amount of oil remaining in the wash solution of Examples 7D and 7E was determined in the following manner. A solid phase extraction (SPE) was carried out on these aqueous samples. Three 20 mL Supelclean™ ENVI-18 SPE tubes (Supelco, Bellefonte, Pa., USA) were placed on a Supelco Preppy SPE manifold (Supelco, Bellefonte, Pa., USA). The tubes were conditioned with approximately 15 mL of 2-propanol followed by 20 mL of distilled water. Then the wash solutions from the Wheaton jars, were added to respective SPE tubes. When the aqueous samples passed through the SPE tube, oil was retained on the column while surfactant and water were not retained on the column. Accordingly, an aqueous surfactant solution was collected from the SPE tube's exit port. The SPE tube was then washed by passing an 80:20 methanol:water solution through it, followed by 15 mL of methanol. The tubes (bearing retained oil) were then left under reduced pressure for 5 minutes to dry. The Wheaton jars were rinsed with 1:1 dichloro-methane:hexanes solution, and the solution was dried over MgSO₄ and added to the SPE tube. The oil remaining on the column was then eluted by passing a 1:1 dichloro-methane:hexanes solution through the SPE tube. Eluate was collected into a pre-weighed 100 mL roundbottom flask. The dichloromethane and hexanes were removed from the flask by rotary evaporation. The roundbottom flask was then weighed, and the difference in mass provided the mass of oil that had been in the aqueous solution. This procedure was performed to determine all traces of oil in the sand washing procedure. As shown in Table 8, room temperature washing of contaminated sand was less effective than washing at 50° C. Accordingly, residual oil in the wash solution was higher for the room temperature procedure relative to the higher temperature procedure.

### Example 7H

### CO₂ Efficiency: Analysis of First Decant in the Absence and Presence of CO₂

The first decanted solution of Examples 7D and 7E were analyzed to determine their mass of oil and to analyze the efficiency of CO₂ in regards to separation of oil from the mixture. A 2 mL sample of first decanted solution (note: no exposure to CO₂, therefore surfactant is ON) was withdrawn and was analyzed per the procedure outlined in Example 7G using a 3 mL Superclean™ ENVI-18 SPE tubes conditioned by 4 mL of 2-propanol followed by 6 mL of distilled water. During the extraction the SPE tube was washed by passing an 80:20 methanol:water solution through it, followed by 2 mL of methanol. Eluates were collected in 50 mL round bottom flasks. Results of this analysis appear in accompanying Table 8. After removal of the sample, CO₂ was bubbled through the first decanted solutions for 10 minutes and then the capped vials were left to stand overnight in the closed CO₂ atmosphere. By exposure to CO₂, surfactant compounds of the invention were switched to their OFF form. The following day, a layer of oil was visible on top of the aqueous mixture. In preliminary studies at 50° C., it was determined that the amount of oil in this layer of oil was 62% of the oil that had originally been added to the sand. It is expected that by optimizing procedures and obtaining further oil from the aqueous layer, it will be possible to increase the amount of recaptured oil.

For the particular switchable surfactant in this study, the aqueous layer appeared as a suspension due to low solubility of the OFF form surfactant in water. A 2 mL sample was withdrawn from the middle of the aqueous mixture layer. Following removal of 2 mL by syringe, the full syringe's needle was manually wiped using a KIMWIPE™ to prevent contamination of the aqueous sample by any oily residue remaining on the syringe needle's tip from penetrating the oily top layer. A solid phase extraction was carried out on this sample as per the procedure outlined in Examples 7G and 7H. Results of this procedure for 2 mL samples of the aqueous layer of first decanted solution in the presence of OFF surfactant are presented in accompanying Table 9.

Notably, substantially less oil remained in the 1^{st} decant solution following treatment with CO₂. As shown in Tables 8 and 9, the amount of oil that remained in the 1^{st} decant when the surfactant was ON was five times the amount of oil that remained in the 1^{st} decant when the surfactant was OFF and time for separation had been provided. The total amount of oil recaptured from the OFF surfactant solution includes both the oil in the oil layer and oil from the aqueous layer.

All publications listed and cited herein are incorporated herein by reference in their entirety. It will be understood by those skilled in the art that this description is made with reference to certain preferred embodiments and that it is possible to make other embodiments employing the principles of the invention which fall within its spirit and scope as defined by the claims.

TABLE 1
Treatment of vials

| Vial | Switching process | | | | | | | | | | | |
|---|---|---|---|---|---|---|---|---|---|---|---|---|
| 1 | ON | | | | | | | | | | | |
| 2 | ON | OFF | | | | | | | | | | |
| 2* | ON | OFF | | | | | | | | | | |
| 3 | ON | OFF | ON | | | | | | | | | |
| 4 | ON | OFF | ON | OFF | | | | | | | | |
| 5 | ON | OFF | ON | OFF | ON | | | | | | | |
| 6 | ON | OFF | ON | OFF | ON | OFF | | | | | | |
| 7 | ON | OFF | ON | OFF | ON | OFF | ON | | | | | |
| 8 | ON | OFF | ON | OFF | ON | OFF | ON | OFF | | | | |
| 9 | ON | OFF | ON | OFF | ON | OFF | ON | OFF | ON | | | |
| 10 | ON | OFF | ON | OFF | ON | OFF | ON | OFF | ON | OFF | | |
| 11 | ON | OFF | ON | OFF | ON | OFF | ON | OFF | ON | OFF | ON | |
| 12 | ON | OFF | ON | OFF | ON | OFF | ON | OFF | ON | OFF | ON | OFF |

TABLE 2
(Method A)

| Vial | Proton integration at ~7.3 δ^{a} |
|---|---|
| 1 | 24 |
| 2 | 0 |
| 3 | 22 |
| 4 | 0 |
| 5 | 20 |
| 6 | 0 |
| 7 | 23 |
| 8 | 0 |
| 9 | 23 |
| 10 | 0 |
| 11 | 24 |

^{a}relative to (CH₃)₄NI set to 100

TABLE 3
(Method B)

| Vial | Proton integration at ~7.3 δ^{b} |
|---|---|
| 1 | 46 |
| 2 | 0 |
| 2* | 45 |
| 3 | 45 |
| 4 | 0 |
| 5 | 43 |
| 6 | 0 |
| 7 | 41 |
| 8 | 0 |
| 9 | 49 |
| 10 | 0 |
| 11 | 41 |
| 12 | 0 |

^{b}relative to (CH₃)₂CHO set to 100

TABLE 4
Surface tension measurement data for vials 1-12 of Table 1

| | Average surface tension | Deviation from the |
|---|---|---|
| Vial | (dynes/cm) | average value^{c} |
| 1 | 35 | +1 |
| 2(2*) | 66(35) | +5 |
| 3 | 33 | −1 |
| 4 | 63 | +2 |
| 5 | 34 | 0 |
| 6 | 63 | +2 |
| 7 | 35 | +1 |
| 8 | 61 | 0 |
| 9 | 34 | 0 |
| 10 | 57 | −4 |
| 11 | 35 | +1 |
| 12 | 56 | −5 |

^{c}Average surface tension in ON and OFF forms being 34 and 61 dynes/cm respectively.

TABLE 5
pH studies of ON and OFF surfactant solutions

| | pH of | | |
|---|---|---|---|
| | Original stock | pH when | pH when |
| Solutions | solution (ON) | turned OFF | turned ON |
| compound (1) in its anionic | 10.87 | 8.02 | 8.78 |
| form (generated by mixing | | | |
| 0.01 M compound (1) | | | |
| in its neutral form and | | | |
| 0.08 M Na₂CO₃) | | | |
| Control (0.08 M solution | 11.42 | 6.78 | 8.98 |
| of Na₂CO₃) | | | |
| Distilled water | 7.00 | 4.50 | 6.75 |

TABLE 6
pH of Surfactant solutions

| | State of | | Deviation of pH from |
|---|---|---|---|
| Vial | the Surfactant | pH of the solution | the average value |
| 1 | ON | 10.85 | — |
| 2 | OFF | 7.35 | −0.38 |
| 3 | ON | 9.38 | +0.07 |
| 4 | OFF | 7.83 | +0.10 |
| 5 | ON | 9.26 | −0.05 |
| 6 | OFF | 7.88 | +0.15 |
| 7 | ON | 9.24 | −0.07 |
| 8 | OFF | 7.70 | −0.03 |
| 9 | ON | 9.22 | −0.09 |
| 10 | OFF | 7.89 | +0.16 |
| 11 | ON | 9.44 | +0.13 |

TABLE 7
Amount of oil removed from sand

| | Room | | |
|---|---|---|---|
| | Temperature | 50° C. | 50° C. |
| | Washing | Washing | Washing |
| | 0.5 wt % | 0.5 wt % | 0.1 wt % |
| | surfactant | surfactant | surfactant |
| Surfactant | solution | solution | solution |
| Sodium octyl 4-hydroxy-3- | 82% | 94-97% | 94% |
| nitrobenzoate | | | |
| Triton X-100 | 68% | 94% | N/A |
| Sodium Dodecyl Sulfate | 40% | 86% | N/A |
| (SDS) | | | |

N/A = not available

TABLE 8
Amount of oil present in 2 mL sample of 1^{st} decant In
the absence of CO₂

| | Room | | |
|---|---|---|---|
| | Temperature | 50° C. | 50° C. |
| | Washing | Washing | Washing |
| | (0.5 wt % | (0.5 wt % | (0.1 wt % |
| | surfactant | surfactant | surfactant |
| | solution) | solution) | solution) |
| Surfactant | (mg) | (mg) | (mg) |
| Sodium octyl 4-hydroxy-3- | 5.2 | 8.8-9.5 | 13 |
| nitrobenzoate | | | |
| Triton X-100 | 11.5 | 22.8 | N/A |
| SDS | 3.4 | 19.7 | N/A |

TABLE 9
Amount of oil present in 2 mL sample of 1^{st} decant after
exposure to CO₂ and time for separation

| | Room | | |
|---|---|---|---|
| | Temperature | 50° C. | 50° C. |
| | Washing | Washing | Washing |
| | 0.5 wt % | 0.5 wt % | 0.1 wt % |
| | surfactant | surfactant | surfactant |
| | solution | solution | solution |
| Surfactant | (mg) | (mg) | (mg) |
| Sodium octyl 4-hydroxy- | 1.0 | 0.5-0.6 | 0.9 |
| 3-nitrobenzoate | | | |

1. A compound comprising
a hydrophobic moiety and a heteroatom;
wherein when an appropriate trigger is applied, the compound in aqueous solution reversibly switches between two states, a neutral state and an anionic state, that are distinguishable from one another by their surface activities;
where the heteroatom is protonated in the neutral state, and the heteroatom is deprotonated and negatively charged in the anionic state; and
wherein a first said trigger, for converting the neutral state to the anionic state in an aqueous solution that has little or no dissolved CO₂, is addition of a base to the aqueous solution; a second said trigger, for converting the neutral state to the anionic state in an aqueous solution that comprises dissolved CO₂, is depletion of CO₂ from the aqueous solution; and a third said trigger, for converting the anionic state to the neutral state, is the addition of CO₂ to the aqueous solution.

2. The compound of claim 1, wherein depletion of CO₂ from the aqueous solution is obtained by: heating the aqueous solution; exposing the aqueous solution to air; exposing the aqueous solution to a gas or gases that has insufficient CO₂ content to convert the anionic state to the neutral state; flushing the aqueous solution with a gas or gases that has insufficient CO₂ content to convert the anionic state to the neutral state; or a combination thereof.

3. The compound of claim 1, wherein the anionic state further comprises a cationic counterion, which is Na⁺, K⁺, Rb⁺, Cs⁺, Li⁺, a transition metal ion, Hg⁺, U⁺, Cr⁺, Pb⁺, Pu⁺, NH₄⁺, NR₄⁺, NRH₃⁺, NR₂H₂⁺, or NR₃H⁺, where R is a lower alkyl group.

4. The compound of claim 1, wherein the hydrophobic moiety comprises a C₄-C₁₀₀ hydrocarbon chain.

5. The compound of claim 4, wherein the hydrocarbon chain is substituted.

6. The compound of claim 1, wherein the heteroatom is O, S, or Se.

7. The compound of claim 1, wherein the compound comprises a hydrophobic moiety that is a hydrocarbon chain, a headgroup attached at one end of the hydrocarbon chain, and a heteroatom that is proximal to the headgroup.

8. The compound of claim 7, wherein the headgroup is an aryl moiety.

9. The compound of claim 8, wherein the aryl moiety is a heteroaryl moiety comprising the heteroatom.

10. The compound of claim 8, wherein the headgroup is an aryl moiety that is substituted by the heteroatom or by a moiety including the heteroatom.

11. The compound of claim 8, wherein the aryl group is substituted by a —((C₁-C₄)-heteroatom) or a -(heteroatom-(C₁-C₄)) moiety.

12. The compound of claim 1, wherein the neutral state comprises a phenol and the anionic state comprises a phenolate.

13. The compound of claim 1, wherein the compound in its neutral state is octyl 4-hydroxy-3-nitrobenzoate.

14. The compound of claim 1, wherein the compound in its anionic state is 2-nitro-4-(octyloxycarbonyl)phenolate.

15. An anionic surfactant comprising:
a hydrophobic moiety and a heteroatom;
wherein when an appropriate trigger is applied, the anionic surfactant in an aqueous solution reversibly switches between two states, an anionic surfactant state and a neutral state, that are distinguishable from one another by their surface activities;
where the heteroatom is protonated in the neutral state, and the heteroatom is deprotonated and negatively charged in the anionic state; and
wherein a first said trigger, for converting the anionic state to the neutral state, is addition of CO₂ to the aqueous solution; a second said trigger, for converting the neutral state to the anionic state in an aqueous solution that has little or no dissolved CO₂, is addition of a base to the aqueous solution; a third said trigger, for converting the neutral state to the anionic state in an aqueous solution that comprises dissolved CO₂, is depletion of CO₂ from the aqueous solution.

16. The anionic surfactant of claim 15, wherein the protonated heteroatom has a pKa that is within about 2.5 pH units of the pKa of H₂CO₃, which is 6.35.

17. The anionic surfactant of claim 15, wherein the neutral state is a demulsifier.

18. A method for stabilizing an emulsion of an aqueous liquid and a water-immiscible liquid comprising:
combining an aqueous liquid and a water-immiscible liquid to form a mixture;
adding to the mixture a compound of claim 1 which is either (i) in its anionic state; or (ii) in its neutral state and is converted to its anionic state in situ; and
agitating the mixture to form a stable emulsion.

19. The method of claim 18, wherein the compound of claim 1 in its anionic state is 2-nitro-4-(octyloxycarbonyl)phenolate (1A) and in its neutral state is octyl 4-hydroxy-3-nitrobenzoate (1N).

20. A method for breaking an emulsion in a mixture, comprising:
providing an emulsion of an aqueous liquid and a water-immiscible liquid, the emulsion including a compound of claim 1 in its anionic state; and
exposing the emulsion to CO₂ to convert the compound from its anionic state to its neutral state.

21. The method of claim 20, further comprising separating the aqueous liquid from the water-immiscible liquid.

22. The method of claim 20, further comprising:
mixing the compound in its neutral state with an aqueous liquid that comprises dissolved CO₂;
eliminating substantially all CO₂ from the mixture; and
reforming the anionic state of the compound.

23. The method of claim 20, wherein the aqueous liquid further comprises a base.

24. The method of claim 23, wherein the base is NaOH, NaHCO₃, or Na₂CO₃.

25. The method of any one of claims 20 to 24, wherein the compound of claim 1 in its anionic state is 2-nitro-4-(octyloxycarbonyl)phenolate (1A).

26. The compound of claim 10, wherein the heteroatom is attached directly to the aryl moiety.

27. A method of remediating soil that is contaminated with one or more hydrophobic chemicals, comprising:
contacting the contaminated soil with a liquid that comprises water and an anionic surfactant of claim 1 so that at least a portion of the hydrophobic chemical becomes associated with the liquid to form contaminated liquid;
optionally separating the contaminated liquid from residual solid soil;
contacting the contaminated liquid with CO₂ to convert a substantial amount of the surfactant from its anionic form to its non-ionic form, resulting in a two-phase liquid mixture having a hydrophobic liquid phase comprising the one or more hydrophobic chemicals, and an aqueous liquid phase;
separating the hydrophobic layer from the aqueous layer.

28. The method of claim 27, wherein the compound in its anionic state is 2-nitro-4-(octyloxycarbonyl)phenolate (1A).

29. A system for cleaning a hydrophobic contaminant from a solid material, comprising:
means for contacting a mixture of solid material and hydrophobic contaminant with a liquid that comprises water and an anionic surfactant of claim 1 so that at least a portion of the hydrophobic contaminant becomes associated with the liquid to form contaminated liquid;
optionally, means for separating the contaminated liquid from residual solid material;
means for contacting the contaminated liquid with CO₂ to convert a substantial amount of the surfactant from its anionic form to its non-ionic form, resulting in a two-phase liquid mixture having a hydrophobic liquid phase comprising the hydrophobic contaminant, and an aqueous liquid phase; and
means for separating the hydrophobic liquid phase from the aqueous liquid phase.
